(19) United States
(12) Patent Application Publication
(10) Pub. No.: US 2005/0288377 A1
(43) Pub. Date: Dec. 29, 2005

(54) Use of calcimimetic as an adynamic bone disease related treatment

(76) Inventor: Thomas L. Cantor, El Cajon, CA (US)

Correspondence Address: MORRISON & FOERSTER LLP, 12531 HIGH BLUFF DRIVE, SUITE 100, SAN DIEGO, CA, 92130-2040, US

(21) Appl. No.: 11/152,550

(22) Filed: Jun. 14, 2005

Related U.S. Application Data
(60) Provisional application No. 60/579,819, filed on Jun. 14, 2004.

(51) Int. Cl. A61K 31/59

(52) U.S. Cl. 514/649

(57) ABSTRACT

The present invention relates to monitoring and treatment of adynamic bone disease and related disorders and the consequences of thereof. In one aspect, the present invention is directed to a method for avoiding or reducing soft tissue calcification (or the risk thereof) in a subject afflicted with adynamic bone disease, using, inter alia, a calcimimetic. In another aspect, the present invention is directed to a kit for avoiding or reducing soft tissue calcification (or the risk thereof) in a subject afflicted with adynamic bone disease, which kit comprises a calcimimetic and a means for assessing bone turnover rate and/or a means for assessing calcium, phosphate and/or calcium x phosphate level.

### CROSS-REFERENCE TO RELATED APPLICATIONS

This application claims the priority benefit of provisional application U.S. Ser. No. 60/579,819, filed Jun. 14, 2004, the content of which is incorporated by reference in their entirety.

### TECHNICAL FIELD

The present invention relates to monitoring and treatment of adynamic bone disease and related disorders and the consequences of thereof.

### DISCLOSURE OF THE INVENTION

In general, calcimimetics bind with the calcium sensing receptor of the parathyroid gland and decrease the sensitivity for receptor activation by extracellular calcium, thus decreasing parathyroid hormone release. Cinacalcet HCl (i.e., AMG-073, available from Amgen, Inc., Thousand Oaks, Calif.) (C₂₂H₂₂F₃N HCl; or N-[1-(R)-(−)-(1-naphthyl)ethyl]-3-[-(trifluoromethyl)phenyl]-1-aminopropane hydrochloride), is an example of a calcimimetic. See, e.g., U.S. Pat. Nos. 6,011,068, 6,031,003, 6,211,244 and 6,313,146. It has been proposed that cincacalcet HCl would be effective in lowering bone turnover. However, recent dual before and after treatment bone biopsy data has indicated that this is not necessarily the case. For example, these data indicate that 28% percent of patients treated with Cinacalcet HCl during a clinical trial witnessed a decrease in bone turnover. See H. H. Malluche, et al., European Renal Association (ERA) 2004 Meeting, Lisbon, Portugal. Notably, the placebo group of the same clinical trial witnessed a lowering of bone turnover in 38% of the group members. See id. Notwithstanding the title of this ERA presentation, these data suggest that cincacalcet HCl does not lower bone turnover to a clinically significant degree. The product insert for Amgen's SENSIPAR™ tablets (Cinacalcet HCl) indicates that this composition decreases both total PTH (comprising, e.g., 1-84 PTH and 7-84 PTH) and calcium, phosphate and the calcium x phosphate product in chronic kidney disease patients on dialysis. See the SENSIPAR™ (Cinacalcet HCl) product insert at the Food and Drug Administration Website, on the World Wide Web at fda.gov/cder/foi/label/2004/21688_sensipar_lbl.pdf This product insert further indicates that use of Cinacalcet HCl carries a risk for developing adynamic bone disease, and if this develops that the use of Cinacalcet HCl should be discontinued.

The whole PTH/7-84 PTH inhibitor ratio (1-84 PTH/7-84 PTH) has been demonstrated to be predictive of the bone turnover rate with greater than 90% accuracy. See A. Tokumoto, J. Amer. Soc. Nephrol. (2003) 14: 702; and M-C Faugere, et al., Kidney Int. (2001) 60: 1460-1468 (indicating and evaluating, in part, biochemical parameters for predicting bone turnover). For example, a decrease in this ratio correlates with a decrease in bone turnover. See, e.g., Publication No. US-2004-0067526-A1. In leading to the present disclosure, we have observed that the administration of vitamin D analogs to a subject actually decreases the 1-84 PTH/7-84 PTH ratio. See J. J. Kazama, et al., Nephrol. Dial. Transplant (2004) 19: 892-7 (indicating that the Intact PTH assay overestimates true 1-84 PTH levels after maxacalcitol therapy in dialysis patients with secondary hyperparathyroidism; see FIG. 3 therein); T. Cantor, et al., Hypocalcemic 7-84 PTH Fragment Level Decreases with a Decrease in Vitamin D Treatment, Abstract submitted for ERA-EDTA Annual Congress, July, 2002, Copenhagen, Denmark (Table 1, reproduced below, indicates that a decrease in Vitamin D led to an increased 1-84PTH/7-84 PTH ratio).

TABLE 1

| | Intact PTH | Ca | PO₄ | | Hectarale | CAP ™ | CIP ™ | |
|---|---|---|---|---|---|---|---|---|
| Dates | pgm/mL | mg/dL | mg/dL | Ca × PO₄ | μgm/dose | pgm/mL | pgm/mL | CAP ™/CIP ™ |
| Aug. 17 | 3559 | 8.3 | 7.0 | 58 | 4 | 1735 | 1824 | 0.95 |
| 2001 | | Sept. 21 | Sept. 21 | | | | | |
| Nov. 1 | 1679 | 8.1 | 5.7 | 46 | 2.5 | 1462 | 217 | 6.74 |
| 2001 | | Nov. 8 | Nov.5-7 | | | | | |

Similarly, it has been observed that cincacalcet HCl does not lower the 1-84 PTH/7-84 PTH ratio. See M. R. Rubin, et al., “A Molecular Form of PTH Distinct From PTH (1-84) is Produced in Parathyroid Carcinoma” (J. Bone & Mineral Metab., forthcoming). Moreover, in leading to the present disclosure, that in parathyroid cancer patients receiving cincacalcet HCl treatment, the 1-84 PTH/7-84 PTH ratio did not decrease; however, there was a significant lowering of serum calcium concentrations (J. Bone & Miner. Met., forthcoming). See Table 2, below.

TABLE 2

| | | | | | | | | | | % DIFF | % DIFF | |
|---|---|---|---|---|---|---|---|---|---|---|---|---|
| Patient # | Visit # | w-PTH | t-PTH | CIP | Ratio | TPTH | CPTH | CIP | Ratio | TPTH | CPTH | CPTH/TPTH |
| 1 | W2 | 946 | 797 | −149 | −6.3 | 684 | 965 | −281 | −3.4 | 14% | −2% | 1.4 |
| 1 | W3 | 936 | 752 | −184 | −5.1 | 729 | 951 | −222 | −4.3 | 3% | −2% | 1.3 |
| 1 | W4 | 962 | 771 | −191 | −5.0 | QNS | QNS | | | | | |
| 1 | WS | 958 | 715 | −243 | −3.9 | QNS | QNS | | | | | |
| 1 | W6 | 915 | 786 | −129 | −7.1 | 413 | 516 | −103 | −5.0 | 47% | 44% | 1.2 |
| 1 | W7 | 1108 | 916 | −192 | −5.8 | 861 | 1059 | −198 | −5.3 | 6% | 4% | 1.2 |
| 1 | W8 | 864 | 750 | −114 | −7.6 | 643 | 821 | −178 | −4.6 | 14% | 5% | 1.3 |
| 1 | W9 | 913 | 805 | −108 | −8.5 | 720 | 869 | −149 | −5.8 | 11% | 5% | 1.2 |
| 1 | W10 | 883 | 786 | −97 | −9.1 | 807 | 853 | −46 | −18.5 | −3% | 3% | 1.1 |
| 1 | W11 | 765 | 747 | −18 | −42.5 | 651 | 698 | −47 | −14.9 | 13% | 9% | 1.1 |
| 1 | W12 | 712 | 662 | −50 | −14.2 | 638 | 733 | −95 | −7.7 | 4% | −3% | 1.1 |
| 1 | W13 | 826 | 720 | −106 | −7.8 | 626 | 759 | −133 | −5.7 | 13% | 8% | 1.2 |
| 1 | W14 | 845 | 795 | −50 | −16.9 | 780 | 825 | −45 | −18.3 | 2% | 2% | 1.1 |
| 1 | W15 | 821 | 733 | −88 | −9.3 | 797 | 822 | −25 | −32.9 | −9% | 0% | 1 |
| 1 | EOT | 868 | 767 | −101 | −8.6 | 630 | 779 | −149 | −5.2 | 18% | 10% | 1.2 |
| 1 | M8 | 993 | 841 | −152 | −6.5 | 876 | 922 | −46 | −20.0 | −4% | 7% | 1.1 |
| 2 | SCR | 330 | 392 | 62 | 5.3 | 379 | 259 | 120 | 2.2 | 3% | 22% | |
| 2 | D1 | 243 | 241 | −2 | −121.5 | QNS | QNS | | | | | |
| 2 | W1 | 220 | 312 | 92 | 2.4 | 253 | 189 | 64 | 3.0 | 19% | 14% | |
| 2 | W2 | 156 | 204 | 48 | 3.3 | 152 | 115 | 37 | 3.1 | 25% | 26% | |
| 2 | W3 | 234 | 300 | 66 | 3.5 | 259 | 166 | 93 | 1.8 | 14% | 29% | |
| 2 | W4 | 131 | 172 | 41 | 3.2 | 165 | 97 | 68 | 1.4 | 4% | 26% | |
| 2 | W5 | 134 | 172 | 38 | 3.5 | 174 | 116 | 58 | 2.0 | −1% | 13% | |
| 2 | M16 | 257 | 155 | 102 | 1.5 | | | | | | | |
| 2 | M24 | 214 | 141 | 73 | 1.9 | | | | | | | |
| 2 | M32 RE | 455 | 284 | 171 | 1.7 | | | | | | | |
| 2 | M32 RE | 613 | 412 | 201 | 2.0 | | | | | | | |
| 2 | M32 RE | 814 | 543 | 271 | 2.0 | | | | | | | |
| 3 | D1 | 319 | 467 | 148 | 2.2 | 478 | 315 | 163 | 1.9 | −2% | 1% | |
| 3 | W1 | 314 | 443 | 129 | 2.4 | 456 | 278 | 178 | 1.6 | −3% | 11% | |
| 3 | W2 | 361 | 497 | 136 | 2.7 | 298 | 210 | 88 | 2.4 | 40% | 42% | |
| 3 | W3 | 306 | 455 | 149 | 2.1 | 411 | 274 | 137 | 2.0 | 10% | 10% | |
| 3 | W4 | 279 | 406 | 127 | 2.2 | 404 | 244 | 160 | 1.5 | 0% | 13% | |
| 4 | SCR | 1619 | 1020 | −599 | −2.7 | 882 | 1345 | −463 | −2.9 | 14% | 17% | 1.5 |
| 4 | D1 | 2227 | 1512 | −715 | −3.1 | 1149 | 1815 | −666 | −2.7 | 24% | 19% | 1.6 |
| 4 | W1 | 2048 | 1352 | −696 | −2.9 | 1050 | 1823 | −773 | −2.4 | 22% | 11% | 1.7 |
| 4 | W2 | 2176 | 1318 | −858 | −2.5 | 945 | 1922 | −977 | −2.0 | 28% | 12% | 2 |
| 4 | W3 | 1373 | 843 | −530 | −2.6 | 717 | 1196 | −479 | −2.5 | 15% | 13% | 1.7 |
| 4 | W4 | 749 | 212 | −537 | −1.4 | QNS | QNS | | | | | |
| 4 | W9 | 775 | 1109 | −334 | −3.3 | 1.4 | | | | | | |
| 4 | W13 | 741 | 1274 | −533 | −2.4 | 1.7 | | | | | | |
| 4 | EOT | 726 | 1454 | −728 | −2.0 | 2.0 | | | | | | |
| 5 | SCR | 225 | 190 | 35 | 5.4 | | | | | | | |
| 5 | W6 | 240 | 177 | 63 | 2.8 | | | | | | | |
| 5 | W9 | 190 | 150 | 40 | 3.8 | | | | | | | |
| 5 | W14 | 142 | 120 | 22 | 5.5 | | | | | | | |
| 5 | N48 | 186 | 160 | 26 | 6.2 | | | | | | | |
| 5 | M16 | 174 | 162 | 12 | 13.5 | | | | | | | |
| 5 | M24 | 229 | 172 | 57 | 3.0 | | | | | | | |
| 6 | SCR | 700 | 830 | −130 | −6.4 | 1.2 | | | | | | |
| 6 | W4 | 477 | 609 | −132 | −4.6 | 1.3 | | | | | | |
| 6 | W7 | 726 | 836 | −110 | −7.6 | 1.2 | | | | | | |
| 6 | W12 | 743 | 860 | −117 | −7.4 | 1.2 | | | | | | |
| 6 | EOT | 805 | 1047 | −242 | −4.3 | 1.3 | | | | | | |
| 6 | M8 | 995 | 1325 | −330 | −4.0 | 1.3 | | | | | | |
| 6 | M16 | 936 | 1512 | −576 | −2.6 | 1.6 | | | | | | |

“SCR” and “D1” represent values prior to Cinacalcet HCl treatment. The remainder of the “visits” are all at different timepoints on Cinacalcet HCl.
W = week (during the first 16 wk titration period); EOT = end of titration phase (the 16 wk visit); M = maintenance week (M1 is 8 weeks after the 16 wk titration period).

Presently, it is suspected that end-stage renal disease (ESRD) patients afflicted with adynamic bone disease are at an increased risk of soft tissue calcification due to the inability of bones to incorporate calcium. See, e.g., M. Eguchi, et al., Am. J. Nephrol. (2000) 20: 278-82 (investigating a method for diagnosing cardiac calcinosis in hemodialysis patients with ESRD, see Table 1 therein); K. Tsuchihashi, et al., Nephron. (2000) 84(1): 13-20 (evaluating the correlation of metabolic calcium abnormalities and hyperparathyroidism with cardiac abnormalities in ESRD patients). It is recognized that soft tissue calcification represents one of the greatest risks to subjects afflicted with adynamic bone disease. As evidence of the detrimental effect of soft tissue calcification, this malady has led to a five to fifteen times greater incidence of myocardial infarction among end stage renal dialysis patients compared to age matched diabetes patients. See Publication No. US-2004-0067526-A1; see also, e.g., P. Raggi, Nephrol. Dial. Transplant (2002) 17: 332-5; W. G. Goodman, Nephrol. Dial. Transplant (2003) 18(Suppl 3): iii2-8; M. Kok, et al., Clin. Nuclear Med. (2003) 28(2): 144-5 (indicating that ESRD patients with secondary hyperparathyroidism may be at risk for metastatic calcification).

It is further recognized that evaluating PTH levels in a subject may provide a useful diagnostic indication in the diagnosis of the risk for soft tissue calcification in the subject. See, e.g., M. Eguchi, et al., Am. J. Nephrol. (2000) 20: 278-82. Relatedly, it is recognized that the ratio of PTH components, e.g., 1-84 PTH/7-84 PTH, will similarly provide a useful diagnostic indication in the diagnosis of the risk for soft tissue calcification in the subject. Often, the ratio will provide a more reliable diagnostic indication for the risk of soft tissue calcification inasmuch as the ratio accurately detects more adynamic bone disease patients (who are at a higher risk of soft tissue calcification) than do low values of intact PTH. This is because the ratio detects adynamic bone disease patients having high levels of intact PTH.

Accordingly, in leading to the present disclosure it was recognized that it is important to control calcium levels, phosphate levels and/or the calcium level x phosphate level product in patients with adynamic bone disease. An increased calcium level, phosphate level and/or calcium level x phosphate level product in a subject may result from excessive administration of certain therapeutic(s) (i.e., vitamin D) which are used to lower bone turnover.

It is further recognized that there are precious few therapeutic options for treating adynamic bone disease, especially with a view of removing adynamic bone disease patients from the risk of developing soft tissue calcification. Accordingly, as cincacalcet HCL (among other calcimimetics) does not appear to lower bone turnover (as indicated in the biopsy study and ratio results cited above), yet it is effective at lowering in vivo calcium, phosphate, and calcium x phosphate levels, it provides a potentially valuable treatment modality useful to decrease the risk of calcification in patients afflicted with adynamic bone disease. As the use of Cinacalcet HCl is contraindicated for subjects having or at risk for developing adynamic bone disease, this represents a novel therapeutic application.

As such, the present disclosure contemplates the use of a calcimimetic such as Cinacalcet HCl in the treatment of a subject afflicted with adynamic bone disease. Frequently, the bone turnover rate may be evaluated as assessed with a PTH ratio or a PTH component (e.g., total PTH, whole PTH, 7-84 PTH, etc.) level. Also frequently, calcium and/or phosphate and/or calcium x phosphate levels are evaluated in the subject with adynamic bone disease who might be treated with a calcimimetic. The subject will often be monitored for bone turnover related indicia and/or calcium and/or phosphate levels before, during and/or after treatment with a calcimimetic such as Cinacalcet HCl.

Accordingly, a method is provided for avoiding or reducing soft tissue calcification (or the risk thereof) in subjects afflicted with adynamic bone disease, comprising administering an effective dose of a calcimimetic, whereby the incidence or extent of soft tissue calcification (or risk thereof) is reduced. The calcimimetic frequently comprises Cinacalcet HCl. The subject is often a subject afflicted with ESRD.

A method is also provided for reducing the incidence of (or the risk thereof) a secondary effect of soft tissue calcification in subjects afflicted with adynamic bone disease comprising administering an effective does of a calcimimetic, whereby the incidence or extent of soft tissue calcification (or risk thereof) is reduced. The calcimimetic frequently comprises Cinacalcet HCl. The secondary effect of soft tissue calcification often comprises a detrimental consequence of soft tissue calcification in a subject, including, for example, an increased risk of myocardial infarction, ectopic or heterotopic calcifications, myositis ossificans, etc. The subject is often a subject afflicted with ESRD.

Unless defined otherwise, all terms of art, notations and other scientific terms or terminology used herein have the same meaning as is commonly understood by one of ordinary skill in the art to which this invention belongs. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art. As appropriate, procedures involving the use of commercially available kits and reagents are generally carried out in accordance with manufacturer defined protocols and/or parameters unless otherwise noted. All patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means “at least one” or “one or more.”

As used herein, “whole parathyroid hormone” or “whole PTH” refers to the complete molecule of PTH or a variant, fragment, derivative or analog thereof. Often this molecule stimulates osteoclast formation, osteoblast formation, bone resorption, stimulation of adenylate cyclase and bone turnover to increase blood calcium levels. 1-84 PTH is an example of whole PTH. For purposes herein, the name “parathyroid hormone (PTH)” is used, although all other names are contemplated. See, e.g., Watson et al., MOLECULAR BIOLOGY OF THE GENE 224 (The Benjamin/Cummings Pub. Co., 4^{th} ed. 1987). Other names of PTH include, for example, parathormone and parathyrin. Whole PTH assay values may be obtained by measuring a sample with a variety of assays. Whole PTH refers to any of a variety of species dependent forms of the PTH molecule. See, e.g., Caetano, A. R., et al., Equus Genome Res. 9(12): 1239-1249 (1999) (horse), U.S. patent application Publication US 2002/0110871 A1 (rat, mouse, bovine, canine, porcine), U.S. Pat. Nos. 6,689,566 and 6,743,590 (human).

As used herein, the terms “total PTH” refers to a combination of whole PTH and PTH fragments in a subject. Alternatively, “total PTH” refers to a combination of PTH agonist and PTH antagonist in a subject. Often this “combination” refers to a measurement of the levels of each of the substituents of the total PTH in a subject. This measurement frequently comprises detecting 1-84 PTH and 7-84 PTH in a sample. Often this measurement comprises detecting 1-84 PTH, 7-84 PTH and another PTH fragment, such as 2-84 PTH or 8-84 PTH, in a sample.

As used herein, the term “sample” refers to anything which may contain an analyte for which an analyte assay is desired. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues comprise an aggregate of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s).

As used herein, the term “subject” is not limited to a specific species or sample type. For example, the term “subject” may refer to a patient, and frequently a human patient. However, this term is not limited to humans and thus encompasses a variety of mammalian species.

As used herein, “disease or disorder” refers to a pathological condition in an organism resulting from, e.g., infection or genetic defect, and characterized by identifiable symptoms.

As used herein, “treatment” means any manner in which the symptoms of a condition, disorder or disease are ameliorated or otherwise beneficially altered. Treatment also encompasses any pharmaceutical use of the compositions herein. Non-pharmaceutical and/or noninvasive “treatment” is also contemplated.

As used herein, “disease or disorder” refers to a pathological condition in an organism resulting from, e.g., infection or genetic defect, and characterized by identifiable symptoms.

In a frequent embodiment, a kit is provided for administering a calcimimetic such as Cinacalcet HCl to a subject afflicted with adynamic bone disease, which kit comprises, in a container, means for administering the calcimimetic. Instructions are optionally included for administration of composition by a physician or by the patient.

In one aspect, the present invention is directed to a method for avoiding or reducing soft tissue calcification (or the risk thereof) in a subject afflicted with adynamic bone disease, which method comprises administering to a subject having, or having the risk of developing, soft tissue calcification, an effective dose of a calcimimetic, whereby the incidence or extent of soft tissue calcification (or risk thereof) in said subject is reduced.

The present method can be used to treat any suitable subject. For example, the subject to be treated can be a mammal. In another example, the subject to be treated can be a human, e.g., an end-stage renal disease (ESRD) patient.

The present method can be used for any suitable purposes. For example, the present method can be used to reduce the incidence of (or the risk thereof) a secondary effect of the soft tissue calcification in the subject afflicted with adynamic bone disease. Any suitable secondary effect of the soft tissue calcification can be reduced by the present method. For example, the secondary effect of the soft tissue calcification to be reduced can be myocardial infarction, ectopic or heterotopic calcification, or myositis ossificans.

Any suitable calcimimetic can be used in the present method. For example, the calcimimetic (or the calcium receptor-active molecule) disclosed in the following patents and patent applications can be used: U.S. Pat. Nos. 5,688,938, 5,763,569, 5,858,684, 5,962,314, 6,001,884, 6,011,068, 6,031,003, 6,211,244 B1 and 6,313,146 B1; AU 1,400,801 A and WO 01/34562 A1. Preferably, the calcimimetic used is Cinacalcet HCl.

The present method can further comprise a step of assessing bone turnover rate in the subject. Any suitable methods for assessing bone turnover rate can be used. For example, the bone turnover rate in the subject can be assessed by assessing the level of a PTH component or a ratio between or among PTH components. See e.g., U.S. Pat. Nos. 6,689,566 B1 and 6,743,590 B1, and U.S. patent application Ser. Nos. US2004/185536 A1, US2004/219598 A1 US2004/229281 A1 and US2005/095631 A1. In one example, the level of a PTH component to be assessed is the level of PTH₁₋₈₄. In anther example, the ratio to be assessed is the PTH₁₋₈₄/PTH₇₋₈₄ ratio. The bone turnover rate can be assessed at any suitable time. For example, the bone turnover rate can be assessed before, during and/or after the calcimimetic administration.

The present method can further comprise a step of assessing calcium, phosphate and/or calcium x phosphate level in the subject. The calcium, phosphate and/or calcium x phosphate level can be assessed by any suitable methods. The calcium, phosphate and/or calcium x phosphate level can be assessed at any suitable time. For example, the calcium, phosphate and/or calcium x phosphate level can be assessed before, during and/or after the calcimimetic administration.

In one example, both the bone turnover rate and the calcium, phosphate and/or calcium x phosphate level can be assessed in the subject.

In another aspect, the present invention is directed to a kit for avoiding or reducing soft tissue calcification (or the risk thereof) in a subject afflicted with adynamic bone disease, which kit comprises a calcimimetic and a means for assessing bone turnover rate and/or a means for assessing calcium, phosphate and/or calcium x phosphate level.

Any suitable calcimimetic can be used in the present kit. For example, the calcimimetic (or the calcium receptor-active molecule) disclosed in the following patents and patent applications can be used: U.S. Pat. Nos. 5,688,938, 5,763,569, 5,858,684, 5,962,314, 6,001,884, 6,011,068, 6,031,003, 6,211,244 B1 and 6,313,146 B1; AU 1,400,801 A and WO 01/34562 A1. Preferably, the calcimimetic used is Cinacalcet HCl.

Any suitable means for assessing bone turnover rate can be used. For example, the bone turnover rate in the subject can be assessed by assessing the level of a PTH component or a ratio between or among PTH components. See e.g., U.S. Pat. Nos. 6,689,566 B1 and 6,743,590 B1, and U.S. patent application Ser. Nos. US2004/185536 A1, US2004/219598 A1 US2004/229281 A1 and US2005/095631 A1. In one example, the level of a PTH component to be assessed is the level of PTH₁₋₈₄. In anther example, the ratio to be assessed is the PTH₁₋₈₄/PTH₇₋₈₄ ratio.

The calcium, phosphate and/or calcium x phosphate level can be assessed by any suitable means.

In one example, the present kit comprises both the means for assessing bone turnover rate and the means for assessing calcium, phosphate and/or calcium x phosphate level.

Other features and advantages of the invention will be apparent from the following description.

The present invention is further described by the following examples. The examples are provided solely to illustrate the invention by reference to specific embodiments. These exemplifications, while illustrating certain specific aspects of the invention, do not portray the limitations or circumscribe the scope of the disclosed invention.

### EXAMPLE

Table 3 provides the results of a study of several ESRD subjects treated with Cinacalcet and HECTOROL™ (Doxercalciferol synthetic Vitamin D). Pre and post treatment levels of total PTH, 1-84 PTH, 7-84 PTH, calcium, phosphate and alkaline phosphatase are provided together with the pre and post treatment ratio of 1-84 PTH/7-84 PTH and the pre and post treatment product of (calcium) x (phosphate).

TABLE 3

| | Date | Dose | Dose | Pre- | Post- | Pre- | Post- | Pre- | Post | Pre- | Post- |
|---|---|---|---|---|---|---|---|---|---|---|---|
| Patient | started | cinacalcet | hectorol | Total | Total | CAP | CAP | CIP | CIP | ratio | ratio |
| 1 | 4/17/04 | 30 mg | 0.5 | 1512 | 1573 | 790 | 868 | 722 | 805 | 1.1 | 1.1 |
| 2 | 4/20/04 | 30 mg | 1 | 881 | 560 | 435 | 399 | 245 | 171 | 1.8 | 2.3 |
| 3 | 4/28/04 | 30 mg | 4 | 1445 | 1268 | 811 | 637 | 634 | 631 | 1.3 | 1 |
| | | | (Zemplar) | | | | | | | | |
| 4 | 4/30/04 | 30 mg | 0.5 | 979 | 865 | 581 | 441 | 398 | 424 | 1.5 | 1 |
| 5 | 4/30/04 | 30 mg | 0.5 | 1379 | 1096 | 638 | 498 | 741 | 599 | 0.8 | 0.8 |
| 6 | 4/30/04 | 30 mg | 2 | 673 | 604 | 495 | 399 | 177 | 205 | 2.8 | 1.9 |
| 7 | 4/30/04 | 30 mg | 1 | 710 | 314 | 488 | 188 | 223 | 126 | 2.2 | 1.5 |
| 8 | 5/5/04 | 30 mg | 0.5 | 779 | 774 | 439 | 488 | 340 | 285 | 1.3 | 1.7 |
| 9 | 4/30/04 | 30 mg | 0 | 427 | 519 | 275 | 352 | 152 | 168 | 1.8 | 2.1 |
| 10 | 4/30/04 | 30 mg | 4 | 406 | 712 | 285 | 470 | 122 | 242 | 2.3 | 1.9 |
| 11 | 5/10/04 | 30 mg | 2 | 411 | 351 | 308 | 267 | 105 | 84 | 1.6 | 3.2 |
| | | | 1.0 | 864.0 | 800.4 | 508.8 | 458.3 | 355.3 | 342.2 | 1.7 | 1.7 |
| | Date | Dose | Dose | Pre | Post | Pre | Post | Pre | Prot | Pre alk | Post alk |
| Patient | started | cinacalcet | hectorol | Ca | Ca | Phos | Phos | Ca xP | Ca xP | phos | phos |
| 1 | 4/17/04 | 30 mg | 0.5 | 10.3 | 9 | 6.3 | 7.3 | 65 | 66 | 241 | 397 |
| 2 | 4/20/04 | 30 mg | 1 | 8.8 | 8.7 | 6.5 | 4.9 | 57 | 43 | 288 | 242 |
| 3 | 4/28/04 | 30 mg | 4 | 9 | 9.1 | 6.1 | 5.5 | 55 | 50 | 211 | 242 |
| | | | (Zemplar) | | | | | | | | |
| 4 | 4/30/04 | 30 mg | 0.5 | 10 | 8.2 | 7.4 | 5.4 | 74 | 44 | 453 | 454 |
| 5 | 4/30/04 | 30 mg | 0.5 | 9.4 | 9.2 | 6.3 | 5.7 | 59 | 52 | 344 | 378 |
| 6 | 4/30/04 | 30 mg | 2 | 8.5 | 7.9 | 4.4 | 4.5 | 37 | 36 | 78 | 88 |
| 7 | 4/30/04 | 30 mg | 1 | 8.7 | 8.5 | 5.4 | 5.4 | 47 | 45 | 72 | 72 |
| 8 | 5/5/04 | 30 mg | 0.5 | 9.4 | 8.3 | 4.8 | 4.3 | 45 | 36 | 369 | 424 |
| 9 | 4/30/04 | 30 mg | 0 | 9.2 | 8 | 7.3 | 6.5 | 67 | 52 | 86 | 95 |
| 10 | 4/30/04 | 30 mg | 4 | 10.4 | 8.4 | 5.2 | 5.1 | 54 | 43 | 95 | 110 |
| 11 | 5/10/04 | 30 mg | 2 | 9.9 | 9 | 4.3 | 6.2 | 43 | 56 | 277 | 245 |
| | | | 1.0 | 9.5 | 8.7 | 5.9 | 5.6 | 55.7 | 49.2 | 220.2 | 240.5 |

The above examples are included for illustrative purposes only and are not intended to limit the scope of the invention. Many variations to those described above are possible.

Citation of the above publications or documents is not intended as an admission that any of the foregoing is pertinent prior art, nor does it constitute any admission as to the contents or date of these publications or documents.

1. A method for avoiding or reducing soft tissue calcification (or the risk thereof) in a subject afflicted with adynamic bone disease, which method comprises administering to a subject having, or having the risk of developing, soft tissue calcification, an effective dose of a calcimimetic, whereby the incidence or extent of soft tissue calcification (or risk thereof) in said subject is reduced.

2. The method of claim 1, wherein the subject is a mammal.

3. The method of claim 1, wherein the subject is a human.

4. The method of claim 3, wherein the human is an end-stage renal disease (ESRI)) patient.

5. The method of claim 1, which is used to reduce the incidence of (or the risk thereof) a secondary effect of the soft tissue calcification in the subject afflicted with adynamic bone disease.

6. The method of claim 5, wherein the secondary effect of the soft tissue calcification is selected from the group consisting of myocardial infarction, ectopic or heterotopic calcification and myositis ossificans.

7. The method of claim 1, wherein the calcimimetic is Cinacalcet HCLI.

8. The method of claim 1, further comprises assessing bone turnover rate in the subject.

9. The method of claim 8, wherein the bone turnover rate in the subject is assessed by assessing the level of a PTH component or a ratio between or among PTH components.

10. The method of claim 9, wherein the level of a PTH component to be assessed is the level of PTH₁₋₈₄.

11. The method of claim 9, wherein the ratio to be assessed is the PTH₁₋₈₄/PTH₇₋₈₄ ratio.

12. The method of claim 8, wherein the bone turnover rate is assessed before, during and/or after the calcimimetic administration.

13. The method of claim 1, further comprises assessing calcium, phosphate and/or calcium x phosphate level in the subject.

14. The method of claim 13, wherein the calcium, phosphate and/or calcium x phosphate level is assessed before, during and/or after the calcimimetic administration.

15. The method of claim 1, wherein the bone turnover rate and the calcium, phosphate and/or calcium x phosphate level is assessed in the subject.

16. A kit for avoiding or reducing soft tissue calcification (or the risk thereof) in a subject afflicted with adynamic bone disease, which kit comprises a calcimimetic and a means for assessing bone turnover rate and/or a means for assessing calcium, phosphate and/or calcium x phosphate level.

17. The kit of claim 16, wherein the calcimimetic is Cinacalcet HCl.

18. The kit of claim 16, wherein the means for assessing bone turnover rate comprises a means for assessing the level of a PTH component or a ratio between or among PTH components.

19. The kit of claim 18, wherein the level of a PTH component to be assessed is the level of PTH₁₋₈₄, and the ratio to be assessed is the PTH₁₋₈₄/PTH₇₋₈₄ ratio.

20. The kit of claim 16, which comprises a means for assessing bone turnover rate and a means for assessing calcium, phosphate and/or calcium x phosphate level.
